(19) United States
(12) Patent Application Publication
(10) Pub. No.: US 2003/0114449 A1
(43) Pub. Date: Jun. 19, 2003

(54) SACCHARIN DERIVATIVES AS ORALLY ACTIVE ELASTASE INHIBITORS

(76) Inventors: Peter Aranyi, Budapest Bimbou (HU); Sandor Batori, Budapest (HU); Stephane Dessilla, Canet (FR); Istvan Hermecz, Budapest (HU); Zoltan Kapui, Budapest (HU); Ferenc Levai, Budapest (HU); Endre Mikus, Budapest (HU); Marc Pascal, Dremil-Lafage (FR); Lajos T. Nagy, Budapest (HU); Bruno Simonot, Villeneuve Les Maguelone (FR); Katalin Urban Szabo, Budapest (HU); Marton Varga, Dunakeszi (HU); Lelle Vasvarine Debreczy, Budapest (HU)

Correspondence Address: SANOFI-SYNTHELABO INC., 9 GREAT VALLEY PARKWAY, P.O. BOX 3026, MALVERN, PA, 19355, US

(21) Appl. No.: 10/149,569

(22) PCT Filed: Dec. 14, 2000
(86) PCT No.: PCT/HU00/00130

(30) Foreign Application Priority Data
Dec. 17, 1999 (HU) P9904624

(51) Int. Cl. A61K 31/5377; A61K 31/519; C07D 487/02

(52) U.S. Cl. 514/233.2; 544/116; 544/282; 514/259.41; 514/252.16

(57) ABSTRACT

Orally active compounds of general formula (I) wherein R¹ is methyl, ethyl or 2-morpholino-ethyl group, R² is piperidino, morpholino or 4-methyl-piperazinyl group, n is 2 or 3 and their salts, solvates and hydrates.

This invention relates to the orally active compounds of the general formula (I) useful as elastase-type enzyme inhibitors, for example human leukocyte elastase inhibitors; to their salts, solvates, hydrates of the compounds or their salts, to the pharmaceutical preparations containing these compounds, to the use of the compounds of the general formula (I), to the preparation of the compounds of the general formula (I) and to the new intermediates of the general formula (III) used for the preparation thereof.

It is known from the literature that several groups of compounds have elastase, first of all human leukocyte elastase inhibitory activity. Such type of compounds are for instance peptidyltrifluoromethyl-ketone derivatives, 7-dibromo-cepham derivatives or benzisothiazolone derivatives.

A number of 1,2-benzisothiazole-(1H)-3-one derivatives with in vitro human leukocyte elastase inhibitory effect are disclosed in European patent applications Nos. 626.378 and 483.928 and in J. Med. Chem. 38 (23) p 4687-4692 (1995). Typical representatives of these compounds are the 2-(3-chloro-4-oxo-4H-pyrido[1,2-a]pyrimidin-2-yl)-oxymethyl-4-isopropyl-6-methoxy-1,2-benzisothiazol-(1H)-3-one-1,1-dioxide (EP-0626378A Example 4D.) and the 2-[9-(2-pyrrolidino-ethoxy)-4-oxo-4H-pyrido[1,2-a]pyrimidin-2-yl]-oxymethyl-4-isopropyl-6-methoxy-1,2-benzisothiazol-(1H)-3-one-1,1-dioxide (EP-0626378A Example 9E), for these compounds, however, no oral activity is mentioned in the above literatures or in any other references belonging to the state of the art.

We aimed to find new elastase inhibitory molecules exhibiting high oral inhibitory activity against elastase type—especially human leukocyte elastase type—enzymes, exhibiting good stability, long lasting effect both in vitro and in vivo, high selectivity, good absorption and favourable pharmacochemical, physicochemical characteristics, which render them good candidates for drug development.

We have found that in the case of a 2-(4-oxo-4H-pyrido[1,2-a]pyrimidin-2-yl)-oxymethyl-4-isopropyl-6-substituted-1,2-benzisothiazol-(1H)-3-one1,1-dioxide structure, if we introduce special substituents into position-9 of the pyrido[1,2-a]pyrimidine moiety and methoxy, ethoxy or 2-morpholino-ethoxy group into position-6 of the benzisothiazol moiety, we obtain new enzyme inhibitors possessing very significant oral activity, combined with a number of the desired favourable properties.

The present invention relates namely to the compounds of the general formula (I)—wherein

R¹ stands for methyl group, ethyl group or 2-morpholino-ethyl group;

R² stands for piperidino, morpholino or 4-methyl-piperazino group;

n is 2 or 3—

and to their salts, solvates including hydrates.

Solvates mean the solvates of the compounds of general formula (I) or solvates of a salt of the compounds having general formula (I).

The salt forming partner for the compounds of the general formula (I) may be any pharmaceutically acceptable organic or inorganic compound, e.g. as for racemic or optically active organic compounds: succinic acid, maleic acid, fumaric acid, tartaric acid, citric acid, benzoic acid, mandelic acid and as for inorganic compounds: hydrochloric, hydrobromic, nitric, phosphoric or sulfuric acid.

Especially favourable representatives of the compounds of the general formula (I) contain methoxy group or 2-morpholino-ethyl group for substituent R¹, piperidino, 4-methyl-piperazino or morpholino group for R² and n is 2 or 3.

A further subject of the present invention is the process for the preparation of the compounds of the general formula (I), which comprises reacting a compound of the general formula (II)—wherein the meaning of R¹ is the same as given above, and X stands for a halogen atom—with a compound of the general formula (III)—wherein the meanings of R² and n are the same as given above—and if desired, transforming the resulting compounds of the general formula (I)—wherein the meanings of R¹, R² and n are the same as above—into their salts or liberating them from their salts.

Reaction of the compounds of the general formulae (II) and (III) is preferably carried out in an organic solvent, as for instance dimethylformamide, in the presence of an acid binding agent, favourably in the presence of an organic or inorganic base, as for instance triethylamine, at or above room temperature.

The halogen atom may be fluoro, chloro, bromo or iodo atom.

Preparation of the known compounds of the general formula (II) and of the new compounds of the general formula (III) starts from compounds available on the market, by methods known per se.

By reacting 2-amino-3-hydroxypyridine (Biochem J. 46 p 506-508 (1950)) with 1-(2-halogeno-ethyl)piperidine or 1-(3-halogeno-propyl)morpholine or 2-(4-methyl-piperazino)ethanol the appropriate 3-substituted pyridine was prepared. Reaction of the halogeno derivatives with 2-amino-3-hydroxypyridine was performed in an aqueous-organic solvent mixture, preferably in the presence of a phase transfer catalyst. The substituted alcohols were coupled with 2-amino-3-hydroxypyridine via Mitsunobu-reaction (Organic Reactions/Editor D. Hughes/Vol 42 p 335-656 John Wiley and Sons, New, York 1992).

The resulting 2-amino-3-substituted-pyridines were reacted with an active ester of malonic acid, e.g with its bis-2,4,6-trichorophenyl ester (Monatsch. Chem. 89 S 143-153 (1958)), preferably at elevated temperature, optionally in the presence of phosphoryl chloride, to obtain the 2-hydroxy-9-substituted-4-oxo-4H-pyrido[1,2-a]pyrimidines, the new compounds of the general formula (III)—wherein the meaning of R² and n are the same as given above—as a further subject of the present invention.

The compounds of the general formula (II)—wherein the meaning of R¹ is the same as specified above, X stands for a halogen atom, preferably chloro or bromo atom—were obtained by preparing the 4-isopropyl-6-methoxy- or 4-isopropyl-6-ethoxy-1,2-benzisothiazol-3(2H)-one-1,1-dioxide (J. Med. Chem. 38 (23) p 4687-4692 (1995), Synlett, November 1994 p 933-934)), similarly as described in European Patent Applications Nos. 626378A1 Example 1(b) or 483928A1 Example 5I. and transforming them into the appropriate 2-chloromethyl, or 2-bromomethyl derivatives.

The 2,4-dichloro-benzoyloxy-methyl derivative was prepared according to the method of Example 1A of the latter European patent application. The 2,4-dichloro-benzoyloxy-methyl derivative can also be transformed into the 2-halogenoethyl derivative.

The 6-hydroxy group of the 2-(2,4-dichloro-benzoyloxy-methyl)-4-isopropyl-6-hydroxy-1,2-benzisothiazol-3(2H)-one-1,1-dioxide described in EP-483928A1 Example 1AW can be transformed into heteroarylalkyloxy group.

Further subjects of the present invention are the pharmaceutical products containing the compounds of the general formula (I) and/or the salts, solvates, hydrates thereof, which are preferably products for oral application, but inhalable or parentheral products also form the subject of the invention. The above drug products may be solids or liquids, as for instance tablets, capsules, solutions, suspensions or emulsions. Solid drug product forms, first of all tablets and capsules are preferred. The above drug products are prepared by applying excipients and technological operations conventionally used in the pharmaceutical industry.

The compounds of the general formula (I) according to the present invention are useful for the treatment of diseases whose formation is connected with the liberation, high concentration and proteolytic activity of the elastase enzyme. Such are e.g. inflammatory intestinal diseaseses (irritable bowel distress, irritable bowel syndrome, Crohn-disease, ulcerative colitis), pulmonary hypertension, pediatric broncho-pulmonary dysplasia, rhinitis, chronic obstructive lung disease (COPD), cistitis, cistal fibrosis, acut pancreatitis, hepatitis, immuncomplex mediated III. type immunological inflammation (lupus erythematodes, Goodpasture syndrome, chronic hepatitis, alveolitis), dermatitis, psoriasis, rosacae, vasculitis, IV. type immunological inflammatory reaction (e.g. in the course of tubercolosis, leprosy, Leishmaniosis, Blastomycosis, Schistomiasis), glomerula nephritis, gout arthritis, multiple sclerosis, asthma bronchiale, adult respiratory distress syndrome (ARDS), lack of α₁-antitrypsin, chronic bronchitis, emphysema (including neonatal pulmonary emphysema), pneumonia of neutrofilic origin, surgical intervention, sepsis, trauma, acut inflammations, infections, DIC-syndrome, myocardial infarctus, rheumatoid arthritis and cancer.

Leucocytes, proteolytic enzymes liberated from leucocytes, such as elastases also play essential role in the development of various tissue damages caused by reperfusion appearing after an ischemic event.

Hence, the compounds of the general formula (I) according to the present invention can have significant role in the prevention, treatment and healing of tissue damages caused by reperfusion appearing after an inschemic event.

Further subject of the invention is the use of the compounds of the general formula (I) in the treatment of the diseases listed above.

Content of all publications including but not limited to patents and patent applications cited in this specification are herein incorporated by reference.

It is also be apparent to those skilled in the art that a compound of the general formula (I) can be coadministered with other therapeutic and/or prophylatic agents and/or medicaments that are not medically incompatible therewith.

Compounds of the general formulae (I) and (III), preparation and biological activity thereof are demonstrated by the following examples, without limiting the claims to the examples.

### EXAMPLES

### Example 1

2-amino-3-(2-piperidino-ethoxy)pyridine

110.12 g, (1 mol) of 2-amino-3-hydroxypyridine (Biochem J. 46 p 506-508 (1950)) were dissolved in 500 ml of 40% sodium hydroxide solution. The brown-coloured solution was flushed with argon and 2 g of tetrabutylammonium iodide in 500 ml of dichloro-methane, then 184.11 g, (1 mol) of 1-(2-chloroethyl)piperidine hydrochloride (Chem. Ber. 38 S 3136-3139 (1905)) were added to it, under stirring. The mixture was stirred at room temperature for 5 days, then 500 ml of dichloro-methane and 200 ml of water were added, the phases were mixed well, and separated. The aqueous phase was extracted with 2×150 ml of dichloro-methane, the united organic phase was washed with 3×200 ml of water, dried over magnesium sulfate and evaporated. The reddish-brown crystalline crude product was crystallized from acetone.

Product: 144.71 g (38%) 2-amino-3-(2-piperidino-ethoxy)-pyridine (mp. 105-106° C.)

NMR, δ_{H}(200 MHz, DMSO-D6): 1.37(2H, m, CH₂(CH₂CH₂)₂N), 1.48(4H, m, CH₂(CH₂CH₂)₂N), 2.42(4H, m, CH₂(CH₂CH₂)₂N), 2.64(2H, t, J5.8, NCH₂CH₂O), 4.01(2H, t, J5.8, NCH₂CH₂O), 5.63(2H, s, NH₂), 6.47(1H, dd, J 7.7, 5.0, 5-H), 7.03(1H, dd, J7.7, 1.2, 6-H), 7.51(1H, dd J 5.0, 1.2, 4-H).

### Example 2

2-hydroxy-9-(2-piperidino-ethoxy)-4-oxo-4H-pyrido[1,2-a]pyrimidine

The mixture of 88.5g (0.4 mol) of 2-amino-3-(2-piperidino-ethoxy)pyridine and 550 ml of dry acetone was heated to reflux temperature, then in small portions 185.2 g (0.4 mol) of bis-2,4,6-trichlorophenyl malonate were added to it. Heating was continued for another 1.5 hours, then the mixture was cooled and kept in the refrigerator overnight. The precipitated crystals were filtered off, the mother liquer was concentrated to obtain a second crop, the crops were united, washed with acetone. The resulting crude product was purified by flash chromatography. First the residual 2,4,6-trichlorophenol by dichloro-methane, then the title compound by methanol-dichloro-methane (1:1) mixture was eluated. The latter was evaporated and dried in vacuo.

Product: 69.31 g (60%) 2-hydroxy-9-(2-piperidino-ethoxy)-4-oxo-4H-pyrido[1,2-a]pyrimidine. (mp. 171-172° C.)

NMR δ_{H} (200 MHz, DMSO-D6): 1.39(2H, m, CH₂(CH₂CH₂)₂N), 1.50 (4H, m, CH₂(CH₂CH₂)₂N), 2.50 (4H, m, CH₂(CH₂CH₂)₂N), 2.83(2H, t, J5.9, NCH₂CH₂O) 4.27(2H, t, J5.9, NCH₂CH₂O), 5.16(1H, s, 3-H) 7.13(1H, t, J7.3, 7-H), 7.50(1H, d, J7.3, 8-H) 8.50(1H, d, J6.4, 6-H).

### Example 3

2(-9-(2-piperidino-ethoxy)-4-oxo-4H-pyrido[1,2-a]pyrimidin-2-yl-oxymethyl)-4-isopropyl-6-methoxy-1,2-benzisothiazol-3(2H)-one-1,1-dioxide

57.57 g (0.2 mol) of 2-hydroxy-9-(2-piperidino-ethoxy)-4-oxo-4H-pyrido[1,2-a]pyrimidine were dissolved in 400 ml of dry dimethylformamide, and at room temperature 31 ml of triethylamine and 69.66 g (0.2 mol) 2-bromomethyl-4-isopropyl-6-methoxy-1,2-benzisothiazol-3(2H)-one-1,1-dioxide were added to it. The suspension was stirred under argon atmosphere at room temperature for 6 hours. The suspension was poured onto 1200 ml of ice-water, the crystals were filtered off and crystallized from methanol.

Product: 15.39 g (17%) 2-(9-(2-piperidino-ethoxy)-4-oxo-4H-pyrido[1,2-a]pyrimidin-2-yl-oxymethyl)-4-isopropyl-6-methoxy-1,2-benzisothiazol-3(2H)one-1,1-dioxide (m.p. 138-139° C.).

Elementary analysis:

| C₂₇H₃₂N₄O₇ S | C | H | N | S |
|---|---|---|---|---|
| Calculated | 58.26 | 5.79 | 10.07 | 5.76 |
| Found | 57.41 | 6.02 | 9.77 | 5.39 |

NMR δ_{H} (200 MHz, CDCl3): 1.30(6H, d, J 6.8, (CH₃)₂CH), 1.62(4H, m, CH₂(CH₂CH₂)₂N), 2.64(4H, m, CH₂(CH₂CH₂)₂N), 2.98(2H, t, J 5.8 NCH₂CH₂O), 3.96(3H, s, CH₃O) 4.23(1H, m, J 6.8, (CH₃)₂CH), 4.32(2H, t, J 5.8 NCH₂CH₂O), 5.90(1H, s, 3′-H), 6.23(2H, s, NCH₂O), 7.05(1H, t, J 7.4, 7′-H), 7.14(1H, dd, J 7.7;1.3, 8′-H), 7.19-7.21(2H, m, 5-H, 7-H), 8.72(1H, dd, J 7.0, 1.3, 6′-H).

### Example 4

2(9-(2-piperidino-ethoxy)-4-oxo-4H-pyrido[1,2-a]pyrimidin-2-yl-oxymethyl)-4-isopropyl-6-methoxy-1,2-benzisothiazol-3(2H)-one-1,1-dioxide hydrochloride

1.5 g (2.7 mmol) of 2-(9-(2-piperidino-ethoxy)-4-oxo-4H-pyrido[1,2-a]pyrimidin-2-yl-oxymethyl)-4-isopropyl-6-methoxy-1,2-benzisothiazol-3(2H)-one-1,1-dioxide were dissolved in 100 ml of diethyl ether and 2.5 ml of 20% (m/v) hydrogen chloride in diethyl ether were added to it. The reaction mixture was stirred for 1 hour at room temperature, the resulting crystals were filtered off, dried in exsiccator over sodium hydroxide until constant weight.

Product: 1.55 g (97%) 2-(9-(2-piperidino-ethoxy)-4-oxo-4H-pyrido[1,2-a]pyrimidin-2-yl-oxymethyl)-4-isopropyl-6-methoxy-1,2-benzisothiazol-3(2H)-one-1,1-dioxide hydrochloride (mp. 115-120° C.).

NMR δ_{H} (200 MHz, CDCl3): 1.26(6H, d, J 6.8, (CH₃)₂CHO), 1.71(2H, m, CH₂(CH₂CH₂)₂N), 3.64(4H, m, CH₂(CH₂CH₂)₂N), 2.64(4H, m, CH₂(CH₂CH₂)₂N), 2.98(2H, t, J 5.8 NCH₂CH₂O), 3.96(3H, s, CH₃O) 4.23(1H, m, 6.8, (CH₃)₂CHO), 4.62(2H, t, J 5.8 NCH₂CH₂O), 5.78(1H, s, 3′-H), 6.10(2H, s, NCH₂O), 7.31-7.40(2H, m, 5-H, 7′-H), 7.63(1H, d, 7.0, 8′-H), 7.79(1H, d, J 2.3, 7-H), 8.65(1H, d, J 7.3, 6′-H), 10.26(1H, s).

### Example 5

2-(9-(2-piperidino-ethoxy)-4-oxo-4H-pyrido[1,2-a]pyrimidin-2-yl-oxymethyl)-4-isopropyl-6-methoxy-1,2-benzisothiazol-3(2H)-one-1,1-dioxide-D-monotartarate salt

4.4 g (8.0 mmol) of 2-(9-(2-piperidino-ethoxy)-4-oxo-4H-pyrido[1,2-a]pyrimidin-2-yl-oxymethyl)-4-isopropyl-6-methoxy-1,2-benzisothiazol-3(2H)-one-1,1-dioxide were dissolved in hot methanol (70 ml ) and 1.2 g (8.0 mmol) of D-tartaric acid were added to the solution. The reaction mixture was stirred for 15 min and then cooled down to 10° C.; the resulting crystals were filtered off, washed and dried under vacuum.

Product: 4.63 g (82%) of the title compound (m.p.: 158° C.).

### Example 6

2-(9-(2-piperidino-ethoxy)-4-oxo-4H-pyrido[1,2-a]pyrimidin-2-yl-oxymethyl)-4-isopropyl-6-methoxy-1,2-benzisothiazol-3(2H)-one-1,1-dioxide monofumarate salt

Following the procedure described in Example 5, but using 5.0 g (9.0 mmol) of base and 1.05 g (9.0 mmol) of fumaric acid, as product 4.20 g (69%) of the title compound (m.p.: 183° C.) were obtained.

### Example 7

2-(9-(2-piperidino-ethoxy)-4-oxo-4H-pyrido[1,2-a]pyrimidin-2-yl-oxymethyl)-4-isopropyl-6-methoxy-1,2-benzisothiazol-3(2H)-one-1,1-dioxide benzoate salt

Following the procedure described in Example 5, but using 4.88 g (8.76 mmol) of base and 1.07 g (8.76 mmol) of benzoic acid, as product, finally crystallised in methyl-tert-butylether, 4.00 g (67%) of the title compound (m.p. 114° C.) were obtained.

### Example 8

2-(9-(2-piperidino-ethoxy)-4-oxo-4H-pyrido[1,2-a]pyrimidin-2-yl-oxymethyl)-4-isopropyl-6-methoxy-1,2-benzisothiazol-3(2H)-one-1,1-dioxide monocitrate salt

Following the procedure described in Example 5 but using 7.30 g (13.11 mmol) of base and 2.52 g (13.11 mmol) of citric acid, as product, finally crystallised in methyl-tert-butylether, 10.80 g (quant. Yield) of the title compound (m.p.: 152° C.) were obtained.

### Example 9

2-(9-(2-piperidino-ethoxy)-4-oxo-4H-pyrido[1,2-a]pyrimidin-2-yl-oxymethyl)-4-isopropyl-6-methoxy-1,2-benzisothiazol-3(2H)-one-1,1-dioxide-L-mandelate

Following the procedure described in Example 5, but using 7.00 g (12.58 mmol) of base and 1.91 g (12.58 mmol) of L-mandelic acid, as product, finally crystallised in cyclohexane, 7.60 g (85%) of the title product (m.p.: 102° C.) were obtained.

### Example 10

2-(9-(2-piperidino-ethoxy)-4-oxo-4H-pyrido[1,2-a]pyrimidin-2-yl-oxymethyl)-4-isopropyl-6-methoxy-1,2-benzisothiazol-3(2H)-one-1,1-dioxide 4-isopropyl-6-methoxy-saccharinate salt

55.7 mg of 2-(9-(2-piperidino-ethoxy)-4-oxo-4H-pyrido[1,2-a]pyrimidin-2-yl-oxymethyl)-4-isopropyl-6-methoxy-1,2-benzisothiazol-3(2H)-one-1,1-dioxide were dissolved in 2 ml of methanol and 25.5 mg of 4-isopropyl-6-methoxy-saccharin dissolved in 2 ml of ethanol were added to the solution. The reaction mixture was stirred for 30 minutes at room temperature, the resulting crystals were filtered off, washed with 2×1 ml of cold methanol and dried in vacuum at room temperature.

Product: 45.6 mg (60%) of the title compound (m.p. 203-205° C.).

### Example 11

2-amino-3-(3-morpholino-propoxy)pyridine

1.68 g (0.042 mol) of sodium hydroxide were dissolved in 40 ml of methanol and 4.62 g (0.042 mol) of 2-amino-3-hydroxypyridine were added to it. The mixture was stirred for 20 minutes, then evaporated to dryness. The residue was taken up in 40 ml of methyl sulfoxide and to the mixture 6.91 g (0.042 mol) of 1-(3-chloropropyl)morpholine were added slowly, under cooling with ice-water. The mixture was stirred at room temperature for 18 hours, poured onto 200 ml of ice-water and extracted with 3×30 ml of chloroform. The united organic phase was washed with 5×30 ml of water, dried over anhydrous sodium sulfate and evaporated.

Product: 7.45 g (74%) 2-amino-3-(3-morpholino-propoxy)pyridine (mp.79-81° C.)

NMR, δδ_{H}(²⁰⁰ MHz, CDCl₃): 2.07(2H, m, J 6.2 CH₂CH₂CH₂), 2.44-2.52(6H, m, CH₂N(CH₂CH₂)₂O), 3.72(4H, t, J 4.6, N(CH₂CH₂)₂O),), 4.04(2H, t, J 6.2, CH₂CH₂CH₂O), 4.69(2H, s, NH₂), 6.57(1H, dd, J 7.7, 5.0, 5-H), 6.93 (1H, dd, J 7.7, 1.2, 6-H), 7.77(1H, dd J 5.0, 1.2, 4-H).

### Example 12

2-hydroxy-9-(3-morpholino-propoxy)-4-oxo-4H-pyrido[1,2-a]pyrimidine

From 2-amino-3-(3-morpholino-propoxy)pyridine by following the procedure described in Example 2, crystals of 2-hydroxy-9-(3-morpholino-propoxy)-4-oxo-4H-pyrido[1,2-a]pyrimidine (mp. 200-202° C.) were obtained, yield 63%.

NMR δ_{H} (200 MHz, DMSO-D6): 1.97(2H, m, J4.6 CH₂CH₂CH₂), 2.35-2.52(6H, m, CH₂N(CH₂CH₂)₂O), 3.56(4H, t, J6.4, N(CH₂CH₂)₂O), 4.20(2H, t, J6.4, CH₂CH₂CH₂O), 5.19(1H s, 3-H) 7.18(1H, t, J7.3, 7-H), 7.50(1H, dd, J7.7;0.9, 8-H) 8.51(1H, d, J7.0; 0.9, 6-H).

### Example 13

2-(9-(3-morpholino-propoxy)-4-oxo-4H-pyrido[1,2-a]pyrimidin-2-yl-oxymethyl)-4-isopropyl-6-methoxy-1,2-benzisothiazol-3(2H)-one-1,1-dioxide

From 2-hydroxy-9-(3-morpholino-propoxy)-4-oxo-4H-pyrido[1,2-a]pyrimidine by following the procedure described in Example 3, crystals of 2-(9-(3-morpholino-propoxy)-4-oxo-4H-pyrido[1,2-a]pyrimidin-2-yl-oxymethyl)-4-isopropyl-6-methoxy-1,2-benzisothiazol-3(2H)-one-1,1-dioxide (mp. 76-80° C.) were obtained.

Yield: 17.5%.

Elementary analysis:

| C₂₇H₃₂N₄O₈ S | C | H | N |
|---|---|---|---|
| Calculated | 56.63 | 5.63 | 9.78 |
| Found | 56.57 | 5.76 | 9.56 |

NMR δ_{H} (200 MHz, CDCl₃): 1.30(6H, d, J 6.8, (CH₃)₂CHO), 2.18(2H, m, J6.8 CH₂CH₂CH₂), 2.52(4H, t, J 4.5, N(CH₂CH₂)₂O) 2.67(2H, t, J7.1, NCH₂ CH₂CH₂O), 3.72(4H, t, J4.5, N(CH₂CH₂)₂O), 3.96(3H, s, CH₃O) 4.22-4.28(3H, m, (CH₃)₂CH, NCH₂CH₂CH₂O), 5.91(1H, s, 3′-H), 6.26(2H, s, NCH₂O), 7.08(1H, t, J 7.4, 7′-H), 7.14(1H, dd, J 7.7, 1.3, 8′-H), 7.19 (1H, s, 5-H), 7.26(1H, s, 7-H), 8.72(1H, dd, J 7.0, 1.3, 6′-H).

### Example 14

2-(9-(3-morpholino-propoxy)-4-oxo-4H-pyrido[1,2-a]pyrimidin-2-yl-oxymethyl)-4-isopropyl-6-(2-morpholino-ethoxy)-1,2-benzisothiazol-3(2H)one-1,1-dioxide

From 2-hydroxy-9-(3-morpholino-propoxy)-4-oxo-4H-pyrido[1,2-a]pyrimidine and 2-bromomethyl-4-isopropyl-6-(2-morpholino-ethoxy)-1,2-benzisothiazol-3(2H)on-1,1-dioxide by following the procedure described in Example 3. crystals of 2-(9-(3-morpholino-propoxy)-4-oxo-4H-pyrido[1,2-a]pyrimidin-2-yl-oxymethyl)-4-isopropyl-6-(2-morpholino-ethoxy)-1,2-benzisothiazol-3(2H)-one-1,1-dioxide (mp. 145-150° C.) were obtained. Yield: 13.4%.

Elementary analysis:

| C₃₁H₃₉N₅O₉ S | C | H | N |
|---|---|---|---|
| Calculated | 56.61 | 5.98 | 10.65 |
| Found | 57.41 | 6.34 | 9.59 |

NMR δ_{H} (200 MHz, CDCl₃): 1.30(6H, d, J 6.8 (CH₃)₂CH), 2.18(2H, m, J6.8 CH₂CH₂CH₂), 2.51-2.61(8H, m, 2*N(CH₂CH₂)₂O) 2.69(2H, t, J7.1, NCH₂CH₂CH₂O), 2.85(2H, t, J5.6, NCH₂CH₂O), 3.71-3.77(8H, m, 2*N(CH₂CH₂)₂O), 4.19-4.28(5H, m, (CH₃)₂CH, NCH₂CH₂O, NCH₂CH₂CH₂O), 5.91(1H, s, 3′-H), 6.26(2H, s, NCH₂O), 7.09(1H, t, J 6.5, 7′-H), 7.14(1H, dd, J 6.5, 1.5, 8′-H), 7.21 (1H, s, 5-H), 7.27(1H, s, 7-H), 8.72(1H, dd, J 6.5, 1.5, 6′-H).

### Example 15

2-amino-3-(2-(4-methyl-piperazino)ethoxy)pyridine

To 50 ml of dry tetrahydrofuran under argon 2.6 g (0.02 mol) of 2-(4-methyl-piperazino)ethanol and 6.4 g of triphenylphosphine were added. To the mixture at 0-5° C. 2.2 g (0.02 mol) of 2-amino-3-hydroxypyridine, then dropwise 4.2 g of diethyl azodicarboxylate were added. The reaction mixture, which turnes first to lilac-, then to brown-coloured, was stirred at room temperature for 4 hours, then it was evaporated. The residue was chromatographed on a silicagel coloumn, using dichloro-methane-methanol 19:1 mixture as eluent. The fractions containing the pure product were united and evaporated.

Product: 1.2 g (25%) 2-amino-3-(2-(4-methyl-piperazino)ethoxy)pyridine, as a red-brown oil.

NMR δ_{H} (200 MHz, DMSO-D6): 2.13(3H, s, CH₃N), 2.37(4H, s, CH₃N(CH₂)₂), 2.48(4H, m, (CH₂)₂N CH₂), 2.68(2H, t, J5.8, NCH₂CH₂O), 4.02(2H, t, J5.8, NCH₂CH₂O), 5.60(2H, s, NH₂), 6.47(1H, dd, J 7.7, 5.0, 5-H), 7.02(1H, dd, J7.7;1.2, 6-H), 7.50(1H, dd J 5.0;1.2, 4-H).

### Example 16

2-hydroxy-9-(2-(4-methyl-piperazino)ethoxy)-4-oxo-4H-pyrido[1,2-a]pyrimidine

From 2-amino-3-(2-(4-methyl-piperazino)ethoxy)pyridine by following the procedure described in Example 2, crystals of 2-hydroxy-9-(2-(4-methyl-piperazino)ethoxy)-4-oxo-4H-pyrido[1,2-a]pyrimidine (mp. 180-182° C.) were obtained. Yield 34%.

NMR δ_{H} (200 MHz, DMSO-D6): 2.16(3H, s, CH₃N), 2.34(4H, s, CH₃N(CH₂)₂), 2.50(4H, s, (CH₂)₂NCH₂), 2.76(2H, t, J5.8, NCH₂CH₂O), 4.23(2H, t, J5.8, NCH₂CH₂O), 5.15(1H, s, 3-H) 7.09(1H, t, J7.4, 7-H), 7.44(1H, d, J7.7 8H) 8.48(1H, d, J6.9, 6-H).

### Example 17

2-(9-(2-(4-methyl-piperazino)ethoxy)-4-oxo-4H-pyrido[1,2-a]pyrimidin-2-yl-oxymethyl)-4-isopropyl-6-methoxy-1,2-benzisothiazol-3(2H)on-1,1-dioxide

From 2-hydroxy-9-(2-(4-methyl-piperazino)ethoxy)-4-oxo-4H-pyrido[1,2-a]pyrimidine by following the procedure described in Example 3, crystals of 2-(9-(2-(4-methyl-piperazino)ethoxy)-4-oxo-4H-pyrido[1,2-a]pyrimidin-2-yl-oxymethyl)-4-isopropyl-6-methoxy-1,2-benzisothiazol-3 (2H)-one-1,1-dioxide (mp. 103-106° C.) were obtained. Yield: 19%.

Elementary analysis:

| C₂₇H₃₃N₅O₇ S | C | H | N |
|---|---|---|---|
| Calculated | 56.73 | 5.82 | 12.25 |
| Found | 54.51 | 5.17 | 11.02 |

NMR δ_{H} (400 MHz, DMSO-D6): 1.25(6H, d, J 6.8, (CH₃)₂CH), 2.12(3H, s, CH₃N), 2.33(4H, s, CH₃N(CH₂)₂), 2.61(4H, s, (CH₂)₂N CH₂), 2.84(2H, t, J5.2, NCH₂CH₂O), 3.98(3H, s, CH₃O) 4.11(1H, m, J 6.8, (CH₃)₂CH), 4.29(2H, t, J5.2, NCH₂CH₂O), 5.76(1H, s, 3′-H), 6.11(2H, s, NCH₂O), 7.30(1H, t, 7.4, 7′-H), 7.38 (1H, d, J 1.8, 5-H), 7.53(1H, d, J 7.6. 8′-H), 7.79(1H, d, J 1.8. 7-H), 8.58(1H, d, J 6.9. 6′-H).

### Example 18

2-((2,4-dichloro-benzoyl)oxymethyl)-4-isopropyl-6-(2-morpholino-ethoxy)-1,2-benzisothiazol-3-(2H)-one-1,1-dioxide

To the solution of 0.13 g, (1.0 mmol) of 2-morpholino-ethanol, 0.44 g (1.0 mmol) of 2-((2,4-dichloro-benzoyl)oxymethyl)-4-isopropyl-6-hydroxy-1,2-benzisothiazol-3(2H)-one-1,1-dioxide (EP-483928A1—Example 1A) and 0.22 g (1.2 mmol) of diethyl azodicarboxylate in 10 ml of dry tetrahydrofuran 0.33 g of triphenyl phosphine were added slowly at 0-5° C. under argon atmosphere. The solution was stirred at room temperature for 20 hours, then evaporated in vacuum.

The resulting oil was trituated with abs. ethanol to obtain white crystalline material. The crystals were filtered off, washed with abs. ethanol, dried in vacuum.

Product: 40 mg (72%) of 2-((2,4-dichloro-benzoyl)oxsymethyl)-4-isopropyl-6-(2-morpholino-ethoxy)-1,2-benzisothiazol-3-(2H)-one-1,1-dioxide (mp. 134-136° C.).

NMR δ_{H}(²⁰⁰ MHz, DMSO-D6):, 1.25(6H, d, 6.9, (CH₃)₂CH), 2.48(4H, m, O(CH₂CH₂)₂N), 2.73(2H, t, J 5.5, NCH₂CH₂O), 3.56(4H, m, O(CH₂)₂N, 4.05(1H, m, J 6.9, (CH₃)₂CH), 4.35(2H, t, J 5.5, NCH₂CH₂O), 6.04(2H, s, NCH₂O), 7.38(1H, d, J 2.0, 7-H), 7.58-7.61(3H, m, 3+-H, 4′-H, 5′-H), 7.85(1H, d, J 2.0, 5-H).

### Example 19

2-(bromomethyl)-4-isopropyl-6-(2-morpholino-ethoxy)-1,2-benzisothiazol-3(2H)-one-1,1-dioxide

To the mixture of 1.2 g, (2 mmol) of 2-((2,4-dichloro-benzoyl)oxymethyl)-4-isopropyl-6-(2-morpholino-ethoxy)-1,2-benzisothiazol-3(2H)-one-1,1-dioxide, 5 ml of glacial acetic acid, 0.5 ml of acetic anhydride and at room temperature 6.0 ml of 36 v/w % hydrogen bromide in acetic acid solution were added, the reaction mixture was stirred at 80° C. for 4 hours, then evaporated. The residue was trituated with diethyl ether to obtain white crystalline material. The crystals were filtered off, washed with diethyl ether, dried in vacuum.

Product: 0.75 g (80%) of 2-(bromomethyl)-4-isopropyl-6-(2-morpholino-ethoxy)-1,2-benzisothiazol-3(2H)-one-1,1-dioxide (mp. 192-194° C.)

NMR δ_{H}(²⁰⁰ MHz, CDCl₃): 1.31(6H, d, 6.8, (CH₃)₂CH), 3.60(4H, m, O(CH₂CH₂)₂N), 3.60(2H, t, J 5.5, NCH₂CH₂O), 4.22(4H, m, O(CH₂CH₂)₂N), 4.22(1H, m, J 6.8, (CH₃)₂CH), 4.88(2H, t, J 5.5, NCH₂CH₂O, 5.49(2H, s, NCH₂O), 7.26(1H, d, J 2.0, 7-H), 7.38(1H, d J 2.0, 5-H).

Synthesis of the 2-(9-(2-pyrrolidino-ethoxy)-4-oxo-4H-pyrido[1,2-a]pyrimidin-2-yl-oxymethyl)-4-isopropyl-6-methoxy-1,2-benzisothiazol-3(2H)one-1,1-dioxide, Described in patent application EP-0626378, Example 9E, Used for Comparative Biological Experiments.

0.27 g (1.0 mmol) of 2-hydroxy-9-(2-pyrrolidino-ethoxy)-4-oxo-4H-pyrido[1,2-a]pyrimidine, known from patent application EP 0626378, prepared by the methods described above, were dissolved at room temperature in 5 ml of dry dimethylformamide, to the solution 0.29 ml of triethylamine and 0.32 g (0.9 mmol) of 2-bromomethyl-4-isopropyl-6-methoxy-1,2-benzisothiazol-3(2H)-one-1,1-dioxide were added. The suspension was flushed with argon, stirred at room temperature for 60 hours, then poured onto 200 ml of ice-water. The precipitated crystals were filtered off, crystallized from ethanol, washed with hexane and dried. The crude product was chromatographed on silicagel coloumn, using dichloro-methane-methanol (98:2) mixture eluent. Pure fractions were united and evaporated, the crystals were dried.

Product: 67 mg (5%) of 2-(9-(2-pyrrolidino-ethoxy)-4-oxo-4H-pyrido[1,2-a]pyrimidin-2-yl-oxymethyl)-4-(1-isopropyl)-6-methoxy-1,2-benzisothiazol-3(2H)one-1,1-dioxide (mp. 84-90° C.)

Elementary analysis:

| C₂₆H₃₀N₄O₇ S | C | H | N | S |
|---|---|---|---|---|
| Calculated | 57.55 | 5.57 | 10.33 | 5.91 |
| Found | 52.8 | 5.40 | 8.80 | |

NMR δ_{H}(200 MHz, CDCl₃): 1.30(6H, d, 6.8, (CH₃)₂CH), 1.93(4H, m, (CH₂CH₂)₂N), 2.97(4H, m, (CH₂CH₂)₂N), 3.28(2H, t, J 5.8, NCH₂CH₂O), 3.96(3H, s, CH₃O) 4.21(1H, m, 6.8, (CH₃)₂CH), 4.45(2H, t, J5.8, NCH₂CH₂O), 5.90(1H, s, 3′-H), 6.23(2H, s, NCH₂O), 7.06(1H, t, J 7.4, 7′-H), 7.14-7.27(3H, m, 5-H, 7-H, 8′-H), 8.72(1H, dd, J 7.0. 1.3, 6′H).

The product is identical with the product described in patent application EP 0626378, example 9E.

Strong elastase inhibitory activity of the compounds of the general formula (I) on oral application, is presented by the following experimental results:

Inhibition of Acut Lung Damaging Effect Caused by Human Leukocyte Elastase Enzyme, Determined on Mice.

Description of the method:

Male NMRI mice approximately 6-8 week old of age, weighing between 22-26 grams were dosed per os with the 0.1% (w/v) solution in carboxymethyl cellulose of the investigated compounds of the general formula (I) or of the known comparatory compound, respectively. 60 minutes later the mice were given intratracheally 12.5 international unit of human leukocyte elastase enzyme dissolved in 25.0 μL sterile physiological sodium chloride solution.

3 hours later the animals were euthanized with an overdose of urethane and into the lung 1 ml of physiological salt solution was introduced, for washing.

The trachea was exposed at wound by opening suture clips and a small incision made for insertion of a polyethylene cannula secured in place with surgical thread. An 18 gauge×1½ inch needle, attached to an 1.00 mL syringe was inserted into the cannula, and 0.5 mL of air was withdrawn from the airways. One milliliter was then instilled into the airways. After it the chest was briefly and gently massaged. The syringe was removed from the cannula and the bronchoalveolar lavage (BAL) fluid allowed to drain into a 10.0 mL graduate to determine the total volume of lavage fluid retrievable from lungs while the animal was in a supine position. The instillation procedure mentioned above was repeated three times. Triton X100 was then added to the collected bronchoalveolar lavage fluid (final concentration, 0.2% v/v) to ensure cell disruption and the haemoglobin content was determined spectrophotometrically at 540 nm.

Effectiveness of the elastase enzyme inhibitory compound was determined on the basis of the haemorrhagic responses, according to the following formula:

% inhibition=[(VE−DE)/(VE−VS)]×100,

where:

VE=mean absorbance of BAL fluids from the group pretreated orally only with vehicle but intratracheally with elastase;

DE=absorbance of each BAL fluid from animals pretreated orally with potential inhibitor compound, intratracheally with elastase;

VS=mean absorbance of BAL fluids from group pretreated orally with vehicle and intratracheally with sterile physiological salt solution.

For comparative compound the structurally most related known compound described in patent application EP-0626378A1 example 9E was used. Its synthesis is described above in the reference example.

Experimental results are summarized below, in Table 1.

| | p.o. dose | |
|---|---|---|
| | mg/bw kg | inhibition |
| Compound/Example No. | mice | % |
| 2-(9-(2-piperidino-ethoxy)-4-oxo-4H-pyrido | 10 | 80 |
| [1,2-a]pyrimidin-2-yl-oxymethyl)-4-isopropyl-6- | | |
| methoxy-1,2-benzisothiazol-3(2H)-one-1,1- | | |
| dioxide | | |
| Example 3. | | |
| 2-(9-(3-morpholino-propoxy)-4-oxo-4H-pyrido | 10 | 56 |
| [1,2-a]pyrimidin-2-yl-oxymethyl)-4-isopropyl-6- | | |
| methoxy-1,2-benzisothiazol-3(2H)-one-1,1- | | |
| dioxide | | |
| Example 7. | | |
| 2-(9-(2-(4-methyl-piperazino)ethoxy)-4-oxo-4H- | 10 | 42 |
| pyrido[1,2-a]pyrimidin-2-yl-oxymethyl)-4- | | |
| isopropyl-6-methoxy-1,2-benzisothiazol-3(2H)- | | |
| one-1,1-dioxide | | |
| Example 11. | | |
| 2-(9-(3-morpholino-propoxy)-4-oxo-4H-pyrido | 10 | 70 |
| [1,2-a]pyrimidin-2-yl-oxymethyl)-4-isopropyl- | | |
| 6-(2-morpholinoethoxy)-1,2-benzisothiazol- | | |
| 3(2H)-one-1,1-dioxide | | |
| Example 8. | | |
| Comparative compounds | 10 | 15 |
| 2-(9-(2-pyrrolidino-ethoxy)-4-oxo-4H-pyrido | | |
| [1,2-a]pyrimidin-2-yl-oxymethyl)-4-isopropyl- | | |
| 6-methoxy-1,2-benzisothiazol-3(2H)-one-1,1- | | |
| dioxide | | |
| (EP-626378, Example 9E) | | |

As determined by the above method, the per os ED₅₀ value on mice is 2.6 mg/bw kg in the case of the new 2-(9-(2-piperidino-ethoxy)-4-oxo-4H-pyrido[1,2-a]pyrimidin-2-yl-oxymethyl)-4-isopropyl-6-methoxy-1,2-benzisothiazol-3(2H)-one-1,1-dioxide.

For the known comparative compound (EP-0 626 378 A1, Example 9F) the per os ED₅₀ value on mice is 23 mg/bw kg, as determined by the above method.

It can be seen that the compounds of the general formula (I) of the present invention exhibit strong oral activity, whereas the oral activity of the structurally related known compound is weak.

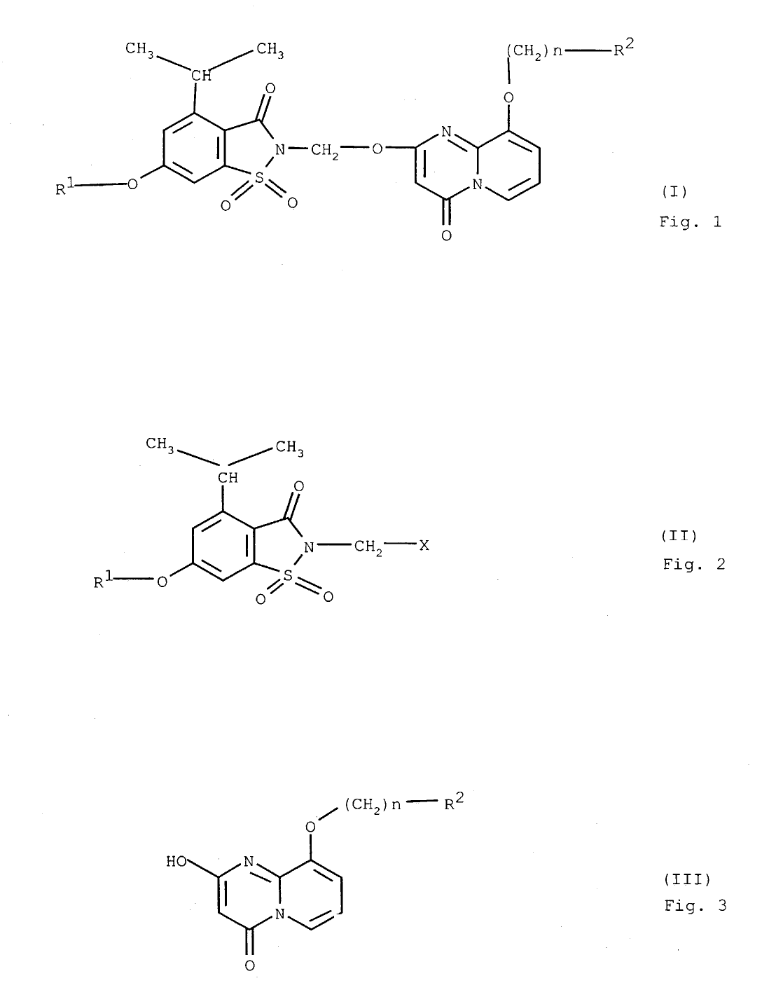

FIG. 1 shows the general formula (I), FIG. 2 shows the general formula (II) and FIG. 3 shows the general formula (III).

1. Compounds of the general formula (I)
wherein
R¹ is methyl, ethyl or 2-morpholino-ethyl-group;
R² is piperidino, morpholino or 4-methyl-piperazinyl group;
n is 2 or 3—
and their salts, solvates and hydrates:

2. Compounds of the general formula (I) according to claim 1
wherein
R¹ is methyl group,
R² and n are as defined in claim 1—
and their salts, solvates and hydrates.

3. Compounds of the general formula (I) according to claim 1
wherein
R² is piperidino group
R¹ and n are as defined in claim 1—
and their salts, solvates and hydrates.

4. 2-[9-(2-piperidino-ethoxy)-4-oxo-4H-pyrido[1,2-a]pyrimidin-2-yl-oxymethyl]-4-isopropyl-6-methoxy-1,2-benzisothiazol-3(2H)-one-1,1-dioxide and its salts, solvates and hydrates.

5. 2-[9-(3-morpholino-propoxy)-4-oxo-4H-pyrido[1,2-a]pyrimidin-2-yl-oxymethyl]-4-isopropyl-6-methoxy-1,2-benzisothiazol-3(2H)-one-1,1-dioxide and its salts, solvates and hydrates.

6. 2-[9-(2-(4-methyl-piperazino)-ethoxy)-4-oxo-4H-pyrido[1,2-a]pyrimidin-2-yl-oxymethyl]-4-isopropyl-6-methoxy-1,2-benzisothiazol-3(2H)-one-1,1-dioxide and its salts, solvates and hydrates.

7. 2-[9-(3-morpholino-propoxy)-4-oxo-4H-pyrido[1,2-a]pyrimidin-2-yl-oxymethyl]-4-isopropyl-6-(2-morpholino-ethoxy)-1,2-benzisothiazol-3(2H)-one-1,2-dioxide and its salts, solvates and hydrates.

8. 2-[9-(2-piperidino-ethoxy)-4-oxo-4H-pyrido[1,2-a]pyrimidin-2-yl-oxymethyl]-4-isopropyl-6-methoxy-1,2-benzisothiazol-3(2H)-one-1,1-dioxide hydrochloride.

9. 2-[9-(2-piperidino-ethoxy)-4-oxo-4H-pyrido[1,2-a]pyrimidin-2-yl-oxymethyl]-4-isopropyl-6-methoxy-1,2-benzisothiazol-3(2H)-one-1,1-dioxide tartarate salts.

10. 2-[9-(2-piperidino-ethoxy)-4-oxo-4H-pyrido[1,2-a]pyrimidin-2-yl-oxymethyl]-4-isopropyl-6-methoxy-1,2-benzisothiazol-3(2H)-one-1,1-dioxide fumarate salts.

11. 2-[9-(2-piperidino-ethoxy)-4-oxo-4H-pyrido[1,2-a]pyrimidin-2-yl-oxymethyl]-4-isopropyl-6-methoxy-1,2-benzisothiazol-3(2H)-one-1,1-dioxide benzoate salt.

12. 2-[9-(2-piperidino-ethoxy)-4-oxo-4H-pyrido[1,2-a]pyrimidin-2-yl-oxymethyl]-4-isopropyl-6-methoxy-1,2-benzisothiazol-3(2H)-one-1,1-dioxide citrate salts.

13. 2-[9-(2-piperidino-ethoxy)-4-oxo-4H-pyrido[1,2-a]pyrimidin-2-yl-oxymethyl]-4-isopropyl-6-methoxy-1,2-benzisothiazol-3(2H)-one-1,1-dioxide mandelate salts.

14. 2-(9-(2-piperidino-ethoxy)-4-oxo-4H-pyrido[1,2-a]pyrimidin-2-yl-oxymethyl)-4-isopropyl-6-methoxy-1,2-benzisothiazol-3(2H)-one-1,1-dioxide 4-isopropyl-6-methoxy-saccharinate salt.

15. A pharmaceutical composition, characterized in that, it contains one or more compounds of the general formula (I)—wherein the meanings of R¹, R² and n are as given in claim 1—and/or their salts together with one or more auxiliary materials applied in the pharmaceutical industry.

16. A pharmaceutical composition, according to claim 15, characterized by, that it contains one or more compounds claimed in claims 4-14 and one or more auxiliary materials applied in the pharmaceutical industry.

17. Pharmaceutical composition suitable for the treatment of syndromes emerging because of elevated elastase concentration, characterized in that, it contains one or more compounds of the general formula (I)—wherein the meanings of R¹, R² and n are as given in claim 1—and/or their salts together with one or more auxiliary materials applied in the pharmaceutical industry.

18. Pharmaceutical composition for the treatment of chronic obstructive pulmonary disease (COPD), adult respiratory distress syndrome (ARDS), pulmonary hypertension, asthma, rheumatoid arthritis, inflammatory bowel disease and cancer according to claim 17, charaterized in that, it contains one or more compounds of the general formula (I)—wherein the meaning of R¹, R² and n are as given in claim 1—and/or their salts, solvates including hydrates together with one or more auxiliary materials applied in the pharmaceutical industry.

19. A pharmaceutical composition according to claim 17, characterized in that, it contains one or more of the compounds claimed in claims 4-14, as for compounds of the general formula (I).

20. The use of the compounds of the general formula (I)—wherein the meanings of R¹, R² and n are as given in claim 1—for the treatment of syndromes emerging because of elevated elastase concentration.

21. The use of the compounds of the general formula (I)—wherein the meanings of R¹, R² and n are as given in claim 1—according to claim 20—for the treatment of chronic obstructive pulmonary disease (COPD), adult respiratory distress syndrome (ARDS), pulmonary hypertension, asthma, rheumatoid arthritis, inflammatory bowel disease and cancer.

22. A process for the preparation of compounds of the general formula (I)—wherein the meanings of R¹, R² and n are as given in claim 1 and their salts—characterized in that, a compound of the general formula (II)—wherein R¹ has the same meaning as given in claim 1, X is a halogen atom, preferably chloro- or bromo atom—is reacted with a compound of the general formula (III)—wherein the meanings of R² and n are as given in claim 1—and the resulted compound of the general formula (I)—wherein the meanings of R¹, R² and n are as given above—, optionally is transformed into its salt or liberated from its salt.

23. A process according to claim 22, characterized in that, a compound suitable for acid binding is used during the reaction.

24. A process according to claim 22, characterized in that, the reaction is carried out in a medium containing organic solvent.

25. A process according to claim 23, characterized in that, organic amines, preferably triethylamine is used as a compound suitable for acid binding.

26. A process according to claim 24, characterized in that, dimethyl-formamide is used as organic solvent.

27. Compounds of the general formula (III)—wherein R² is piperidino, morpholino or 4-methyl-piperazinyl group, n is 2 or 3—and their salts.

28. 2-hydroxy-9-(2-piperidino-ethoxy)-4-oxo-4H-pyrido[1,2-a]pyrimidine.

29. 2-hydroxy-9-(3-morpholino-propoxy)-4-oxo-4H-pyrido[1,2-a]pyrimidine.

30. 2-hydroxy-9-(2-(4-methyl-piperazino)-ethoxy)-4-oxo-4H-pyrido[1,2-a]pyrimidine.

31. The use of compounds of the general formula III or their salts—wherein the meaning of R² and n are the same as in claim 27—for the preparation of compounds of the general formula (I).
